(19) United States
(12) Patent Application Publication
(10) Pub. No.: US 2002/0100075 A1
(43) Pub. Date: Jul. 25, 2002

(54) Transgenic plants with increased expression of VTC4 gene

(76) Inventors: Patricia L. Conklin, Freeville, NY (US); Robert L. Last, Needham, MA (US)

Correspondence Address: BROWN & MICHAELS, PC, 400 M & T BANK BUILDING, 118 NORTH TIOGA ST, ITHACA, NY, 14850, US

(21) Appl. No.: 09/909,600

(22) Filed: Jul. 20, 2001

Related U.S. Application Data
(63) Continuation-in-part of application No. 09/441,318, filed on Nov. 16, 1999.
(60) Provisional application No. 60/126,680, filed on Mar. 29, 1999.

(51) Int. Cl. A01H 5/00

(52) U.S. Cl. 800/278; 800/289

(57) ABSTRACT

The present invention includes a transgenic plant containing a GDP-mannose pyrophosphorylase gene. A pathway for AsA biosynthesis that features GDP-mannose and L-galactose has recently been proposed for plants. A collection of AsA-deficient mutants of Arabidopsis thaliana that are valuable tools for testing of a novel AsA biosynthetic pathway have been isolated. The best characterized of these mutants (vtc1-vitamin c) contains ˜25% of wildtype AsA and is defective in AsA biosynthesis. Using a combination of biochemical, molecular, and genetic techniques, it has been conclusively demonstrated that the VTC1 locus encodes GDP-mannose pyrophosphorylase (mannose-1-P guanyltransferase). This enzyme provides GDP-mannose, which is used for cell wall carbohydrate biosynthesis and protein glycosylation, as well as for AsA biosynthesis.

### REFERENCE TO RELATED APPLICATIONS

This is a continuation-in-part patent application of copending application Ser. No. 09/441,318, filed Nov. 16, 1999, entitled “TRANSGENIC PLANT WITH INCREASED EXPRESSION OF GDP-MANNOSE PYROPHOSPHORYLASE”, which claims the benefit under 35 U.S.C. § 119(e) of Provisional Application No. 60/126,680, filed Mar. 29, 1999, entitled “TRANSGENIC PLANT WITH INCREASED EXPRESSION OF GDP-MANNOSE PYROPHOSPHORYLASE”. The aforementioned applications are hereby incorporated herein by reference.

### ACKNOWLEDGMENT OF GOVERNMENT SUPPORT

This invention was made with Government support under Grant No. 96-35100-3212, awarded by the United States Department of Agriculture. The government has certain rights in the invention.

### BACKGROUND OF THE INVENTION

1. Field of the Invention

The invention pertains to the field of transgenic plants. More particularly, the invention pertains to a transgenic plant expressing a recombinant VTC4 gene.

2. Description of Related Art

Reactive oxygen species (ROS), such as hydrogen peroxide (H₂O₂), superoxide, and the hydroxyl radical, are generated by metabolic processes, chemical compounds (e.g., drugs, pesticides, or carcinogens) that are foreign to the organism, and in response to pathogens in organisms with an aerobic lifestyle. ROS are highly reactive and can oxidize biomolecules, including proteins and nucleic acids. Oxidation of fatty acids has the potential to initiate lipid peroxidation chain reactions. However, controlled oxidative responses appear to play roles in normal biological processes. An example is programmed cell death, defined in animal systems as apoptosis, and exemplified by the hypersensitive response in plants, the localized premature cell death phenomenon that characterizes incompatible pathogen-plant interactions.

ROS are generated by a wide variety of factors in plants. Under normal conditions, ROS are generated during photosynthesis by oxygen photoreduction. High light levels can result in photooxidative damage when ROS production exceeds that of the antioxidant capacity. Such conditions occur when high light is combined with other environmental conditions, such as drought, temperature extremes, or nutrient deprivation. Other factors in the plant's environment also lead to increased ROS, including UV-B, air pollutants (e.g., ozone, sulfur dioxide), redox-active herbicides (e.g., paraquat), and phytotoxic metals (e.g., Zn, Cu, Cd). Plants generate ROS in oxidative bursts that occur during pathogen infection. H₂O₂ generated during oxidative bursts is thought to play an important role in initiation of the hypersensitive response, although the levels of this ROS must be tightly controlled.

As is the case with all organisms, plants have the ability to detoxify ROS. This is accomplished in part with antioxidants including thylakoid-associated α-tocopherol and carotenoids, and soluble molecules such as L-ascorbic acid (Vitamin C), glutathione (and homoglutathione), polyamines and phenolics.

Vitamin C (AsA; L-ascorbic acid) is one of the best-known plant antioxidants. AsA is present in millimolar concentrations in most plant tissues and is a crucial antioxidant and cellular reductant. As an antioxidant, AsA has the capacity to eliminate several different ROS including singlet oxygen, superoxide, and hydroxyl radicals. It also maintains the membrane-bound antioxidant α-tocopherol in the reduced state and is used as a substrate by AsA peroxidase, removing H₂O_{2.}

In addition to its antioxidant capacities, AsA also preserves the activity of a number of enzymes by maintaining prosthetic group metal ions in the reduced state. Although this function of AsA is well known in animal systems, it has not been widely studied in plant systems. In vivo evidence does indicate that AsA is necessary for the activity of the enzyme responsible for conversion of violaxanthin to zeaxanthin during conditions in which excess light energy is dissipated. Plant cell wall expansion and metabolism, as well as cell division, also are thought to depend at least in part on AsA. Finally, AsA can be catabolized to tartrate or oxalate in certain plant species. Given the importance of AsA in these and other roles, and its abundance in all plants tested, it is surprising that its biosynthetic pathway in plants has remained enigmatic. However, significant progress has recently been made towards the understanding of AsA biosynthesis in plants.

### SUMMARY OF THE INVENTION

Vitamin C (L-ascorbic acid) acts as a potent antioxidant and cellular reductant in plants and animals. L-ascorbic acid (AsA) has long been known to have many critical physiological roles in plants, yet its biosynthesis is only currently being defined. A pathway for AsA biosynthesis that features GDP-mannose and L-galactose has recently been proposed for plants. The present invention includes a transgenic plant containing a recombinant VTC1 or VTC4 nucleic acid. The invention also includes an assay for detecting ascorbic acid in plant tissues.

A collection of AsA-deficient mutants of Arabidopsis thaliana that are valuable tools for testing of a novel AsA biosynthetic pathway have been isolated. The best characterized of these mutants (vtc1-vitamin c) contains ˜25% of wild type AsA and is defective in AsA biosynthesis. Using a combination of biochemical, molecular, and genetic techniques, it has been conclusively demonstrated that the VTC1 locus encodes GDP-mannose pyrophosphorylase (mannose-1-P guanyltransferase). This enzyme provides GDP-mannose, which is used for cell wall carbohydrate biosynthesis and protein glycosylation, as well as for AsA biosynthesis.

In an embodiment of the invention, a genetically engineered plant includes a recombinant nucleic acid encoding a protein involved in Vitamin C biosynthesis. This protein preferably is GDP-mannose pyrophosphorylase (encoded by VTC1) or a protein encoded by VTC4.. The genetically engineered plant is capable of producing increased levels of Vitamin C. The plant also possesses increased resistance to environmental stresses compared to wild type plants.

In another embodiment of the invention, a genetically engineered plant includes a recombinant nucleic acid encoding GDP-mannose pyrophosphorylase (encoded by the gene VTC1) or a nucleic acid encoding a protein produced by the gene VTC4. The genetically engineered plant is capable of expressing the recombinant nucleic acid. It can also produce increased levels of Vitamin C. The genetically engineered plant has increased resistance to environmental stresses than wild type plants.

Another embodiment of the invention is a method of increasing the endogenous level of Vitamin C produced in a plant by over-expression of an enzyme crucial to Vitamin C biosynthesis. This enzyme is preferably GDP-mannose pyrophosphorylase (encoded by VTC1) or an enzyme encoded by VTC4. Increasing the endogenous level of Vitamin C leads to increased resistance to environmental stresses.

In another embodiment of the invention, a genetically engineered plant includes a mutant gene that encodes a form of GDP-mannose pyrophosphorylase, or a mutant VTC4 gene.

### BRIEF DESCRIPTION OF THE DRAWING

FIG. 1 shows the proposed Smirnoff-Wheeler pathway for L-ascorbic acid biosynthesis in higher plants.

FIG. 2 shows the amount of ¹⁴C-AsA expressed as a percent of ¹⁴C in the total soluble fraction.

FIG. 3A shows the fine mapping of VTC1 to a position on chromosome 2 to one side of two molecular markers.

FIG. 3B shows the sequence of a 92 kb BAC (T5I7) within the contig of FIG. 3A.

FIG. 3C shows a genomic clone including ˜1.1 kb upstream of the 5′ end of the GDP-mannose pyrophosphorylase cDNA and ˜0.2 kb downstream of the predicted stop codon.

FIG. 3D shows a single cytosine to thymine point mutation at position +64 relative to the first base of the presumed initiator methionine that the vtc1-1 and vtc1-2 mutants contain.

FIG. 4 shows the measurement of GDP-mannose pyrophosphorylase activity in extracts from both vtc1-1 and wild type.

FIG. 5 shows the localization of VTC4 to an approximately 41 kb region at the top of chromosome III of Arabidopsis thaliana.

FIG. 6 shows the localization of VTC4 to an approximately 8.5 kb region contained on BAC F13E7 that includes two predicted Arabidopsis thaliana genes: F13E7.12 and F13E7.13.

FIG. 7 shows the map positions of various VTC loci on the Arabidopsis thaliana genome.

FIGS. 8A and 8B show bar graphs of accumulation of L-galactosyl residues in the cell wall.

FIG. 9 shows a bar graph of the alteration of galactose metabolism in the mutant vtc4-1.

FIG. 10 shows a graph of AsA concentration, based on the NBT assay.

FIG. 11 shows a graph indicating that the VTC2 gene exhibits a gene dosage effect. FIG. 12 shows a graph of the quantitative analysis of total AsA levels in two-week-old vtc mutant and wt leaves.

FIG. 13 shows a graph of AsA in vegetative and reproductive tissues of six-week-old vtc mutants and wt.

### DETAILED DESCRIPTION OF THE INVENTION

Two different plant AsA biosynthetic pathways have been previously proposed; one is similar to the animal pathway, while the other is quite distinct. Animals that synthesize AsA do so via the substrates D-glucose, D-glucuronic acid, L-gulonic acid, and L-gulono-1,4-lactone, which is oxidized to AsA. In the first hypothesized pathway, the carbon skeleton of the primary substrate glucose is inverted in the final product, and this inversion occurs after glucuronate formation. An analogous pathway has been proposed for plants with D-galacturonate and L-galactono-1 ,4-lactone as two key intermediates. However, there are strong radioactive tracer data indicating that inversion of the glucose carbon skeleton does not occur during AsA biosynthesis in higher plants, which would refute the likelihood that these pathways are correct. A non-inversion pathway with the intermediates D-glucosone and L-sorbosone was also proposed. The evidence for this pathway is not very compelling, and no recent data have been published in support of it.

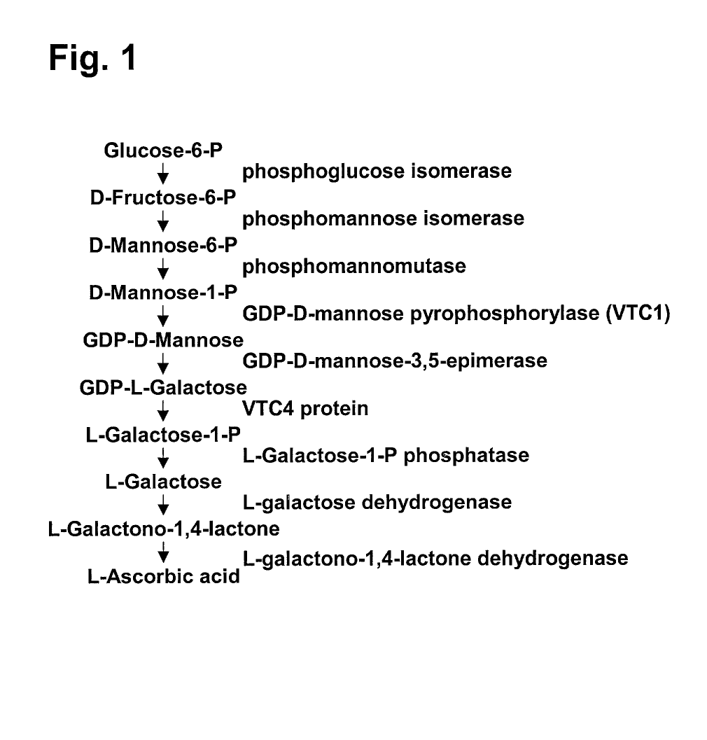

In vitro biochemical methods have recently generated evidence for a novel AsA biosynthetic pathway (FIG. 1) that does not predict inversion of the glucose skeleton, with D-mannose and L-galactose as two key intermediates. Supporting the hypothesis that mannose is a key intermediate in the pathway, when Arabidopsis leaves are fed with [¹⁴C] mannose, 10% of the label appears in AsA by the end of a 4-h incubation. It has also been shown that [¹⁴C] L-galactono-1,4-lactone could be formed when a pea embryo extract was supplied with [¹⁴C] GDP-mannose and NAD. The [¹⁴C] L-galactono-1,4-lactone in vitro-synthesized from [¹⁴C] mannose could subsequently be converted in vitro to [¹⁴C] AsA with the addition of intact mitochondria (to supply GLDH) and cytochrome c as an electron acceptor. It has been proposed that the conversion from GDP-mannose to L-galactono-1,4-lactone proceeds occurs via L-galactose. L-galactose can be synthesized from GDP-mannose by a previously described GDP-D-mannose-3,5-epimerase activity that was detected in both pea and Arabidopsis. A previously undescribed activity (L-galactose dehydrogenase) also detected in these extracts was partially purified and shown to oxidize L-galactose to L-galactono-1,4-lactone, providing substrate for GLDH. A fascinating implication of this pathway is that it plays a key role in plant metabolism; in addition to serving as intermediates for AsA biosynthesis, intermediates in this proposed pathway are also utilized in other metabolic pathways.

Referring to FIG. 1, the proposed AsA biosynthetic pathway has branch points leading to both cell wall and glycoprotein biosynthesis. GDP-mannose is utilized in multiple biosynthetic processes. Both prokaryotes and eukaryotes utilize GDP-mannose in the synthesis of complex structural carbohydrates. GDP-mannose contributes to the synthesis of at least three different structural carbohydrates in plant cell walls. First, hemicellulose polymers, known as mannans, contain D-mannose obtained from its activated form. Secondly, GDP-mannose is the substrate for GDP-D-mannose-4,6-dehydratase, an enzyme that catalyzes the first step in GDP-L-fucose biosynthesis and encoded by the MUR1 gene in Arabidopsis. L-fucose is present in both plant cell walls and glycoproteins. Finally, the proposed intermediate L-galactose is a minor component of the complex carbohydrates found in the non-cellulose portion of the plant cell wall. In addition to a major role in structural carbohydrate biosynthesis, GDP-mannose also has a key eukaryotic role in glycosylation. In eukaryotes, most secretory and membrane proteins are glycosylated. D-mannose, the major carbohydrate component of both N- and O-linked saccharides, is transferred from GDP-mannose during the glycosylation process.

There is little known about AsA biosynthesis. In order to elucidate this process, this invention provides a method for searching for genes involved in AsA biosynthesis (i.e., VTC genes). In order to achieve this goal, mutant plants which are Vitamin C deficient are created. Then, the genes which are affected in these mutants are pinpointed. The sequences of these genes can be determined, and compared to known sequences in a national database. Lastly, the identity of the gene(s) can be verified with the creation of recombinant plants capable of “rescuing” the mutant phenotype (AsA deficiency). By utilizing these techniques, a transgenic plant that can functionally express GDP-mannose pyrophosphorylase has been created. Also, a method of increasing Vitamin C production in a system where GDP-mannose pyrophosphorylase is a limiting factor is disclosed.

Assay for Detection of AsA in Plant Tissues

In order to quickly obtain additional vtc mutants, a direct screen for ascorbate deficiency is used. A quick semi-quantitative assay for the measure of ascorbic acid is described below.

A qualitative AsA assay was developed that utilizes nitroblue tetrazolium (NBT) as a reagent for the visual detection of AsA. This new AsA assay utilizes the electron transfer dye, NBT, which can be reduced by four electrons to yield the dark bluish-purple insoluble formazan. Purified AsA reduces NBT to the formazan, and the high AsA content in plant tissue has allowed us to take advantage of this property.

Arabidopsis leaves ˜3-8 mm in length are excised and laid on a sheet of chromatography paper. Whatman™ 3030-6185 paper (Whatman Ltd., Kent UK) works well for this assay, while generic brands do not. Each leaf is then squashed onto the chromatography paper using a curved metal weighing spatula. Ten μl of a 1 mg/ml aqueous solution of NBT (Sigma, St. Louis, Mo.) is then pipetted directly onto each squashed leaf. Within approximately five minutes, a bluish-purple formazan precipitate is visualized around each wild type leaf. As the formazan tends to bleed through the chromatography paper, this precipitate can often be visualized better on the backside of the paper. Typically, mutant plants do not contain enough ascorbic acid to convert the nitroblue tetrazolium to visible formazan.

The NBT assay was used to directly screen ˜6,000 M₂ plants, and resulted in the identification of six new vtc mutants, one of which was vtc1-2. These mutant plants do not convert the nitroblue tetrazolium to visible formazan, thereby making them deficient in ascorbic acid production.

The vtc mutants described above were identified as having a diminished ability to reduce NBT to formazan. To quantitatively measure the AsA-deficiencies in these mutants, a spectrophotometric method was used to measure total AsA in two-week-old rosettes from each of the vtc mutant lines. The lines used in this analysis have all been back-crossed at least once to the wt progenitor to segregate away unlinked mutations. Our results indicate that the vtc mutants contain one-third to one-half the total AsA present in the wt Col-0 progenitor as shown in FIG. 12.

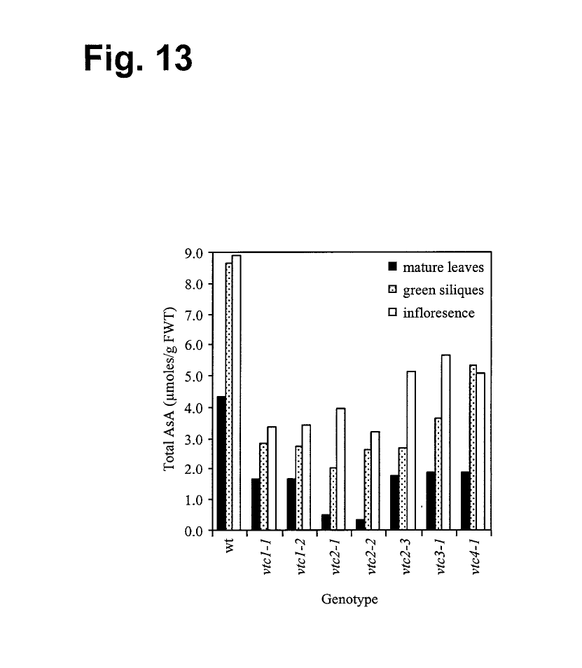

In plants, AsA levels are known to increase upon transition from the vegetative to reproductive state. To determine whether such an increase occurs in the wt and vtc mutants, total AsA was measured in mature rosette leaves, immature green siliques (seed pods), and the inflorescence (containing a mixture of opened and unopened flowers) of six-week-old plants. As shown in FIG. 13, reproductive tissues from wt (green siliques and inflorescences) contain approximately twice the amount of AsA found in wt mature leaves. All the vtc mutants are also able to maintain higher levels of AsA in the reproductive tissues relative to that in leaves. In fact, the floral tissues of these AsA-deficient mutants contain >3 μmoles/g FWT AsA, matching the levels found in rosette leaves of wt.

An interesting result was obtained upon comparison of the AsA levels in the leaves from six-week-old wt and vtc mutants. In mature (fully expanded) leaves, the majority of vtc mutants maintain AsA levels at approximately 40% (˜1.7 μmoles/g FWT) of wt. vtc2-1 and vtc2-2 represent an exception, and mature leaves from these two mutant lines have unusually low levels of AsA (˜10% of wt; ˜0.40 μmoles/g FWT). vtc2-1 is also severely AsA deficient in younger leaves and cauline (stem) leaves from older plants. In summary, six-week-old vtc2-1 and vtc2-2 have a very severe AsA deficiency in leaves, while siliques and inflorescences from these same plants as well as leaves from two-week-old plants are not as severely deficient. This suggests either an underlying difference(s) in AsA metabolism in these different tissue types, or that VTC2 is a regulatory gene.

Creating Plants Mutant in AsA Biosynthesis and Plant Growth Conditions

A plant mutant in a step leading to the biosynthesis of AsA is needed. To create this plant, a mutagenization protocol is performed. The Arabidopsis thaliana used in all of the experiments and the T₁ transgenics were grown in “Cornell Mix” soil (Landry, L. G., Chapple, C. C. S. & Last, R. L. (1995) Plant Physiol. 109, 1159-1166).

All wt and mutant Arabidopsis thaliana lines used in this study are derived from the Columbia (Col-0) ecotype. Mutant lines used for both the quantitative measure of AsA and determination of ozone sensitivity were derived from at least one back-cross to Col-0 wt. The vtc1-1 line was derived from four back-crosses, while the vtc1-2 and vtc4-1 lines were each back-crossed twice.

The T₁ transgenics were grown in a light room (80-100 μmol m⁻² sec⁻¹ light provided by 400 W metal halide bulbs, 20-22° C., 25% relative humidity) under a 16 hour photoperiod. Prior to transformation by vacuum infiltration, plants were grown under a 12 hour photoperiod with other conditions as described by Conklin et al., in Plant Physiol. 109, 203-212 (1995), the complete disclosure of which is hereby incorporated herein by reference.

T₂ transgenics were germinated on sterile plant nutrient medium as described in Li, J. et al. (1995) Plant Cell 7, 447-461, and then transplanted to soil and grown under the same conditions as the T₁ transgenics. The tissues used for the tracer study and GDP-mannose pyrophosphorylase activity assay were from plants grown in a greenhouse in Exeter, U.K. as described by Conklin et al., in Plant Physiol. 115, 1277-1285 (1997), the complete disclosure of which is hereby incorporated herein by reference. All experiments using vtc1-1 were performed on a line that had been back-crossed to the wild type Col-0 progenitor four times.

The Arabidopsis vtc1-1 mutant was isolated from EMS mutagenized Col-0 wild type plants by virtue of its ozone sensitivity. EMS is utilized to induce random point mutations in DNA. vtc1-1 contains ˜25% of wild type AsA concentrations, and results strongly suggest that this deficiency is due to a defect in AsA biosynthesis. This mutant was used as a tool to identify the VTC1 gene.

EMS (ethylene methanesulfonate) is used to induce random point mutations in DNA. Plants arising from this treatment can then be screened for a phenotype of choice (such as, for example, ozone-sensitivity or ascorbate deficiency) to isolate mutants in systems of interest. In the treatment, wild type seeds are soaked in a solution containing EMS, rinsed several times in water, and planted in “pools” consisting of either pots or flats, each containing several thousand seeds. These seeds are known as the M₁ generation (i.e., mutagenesis 1). Mutants in this generation are expected to be heterozygous for the mutation, as the probability of the EMS mutagenizing both chromosomes (of each pair) in exactly the location is extremely low. So, as most mutants of interest are “loss of function” mutants and are recessive, the Ml seed is allowed to grow up, self-pollinate, and produce M₂ seed. If every mutation in the Ml is recessive, one quarter of the resultant M₂ seed (from a single M₁ plant) are expected to be homozygous for the mutation (3:1 Mendellian ratio of wild type to mutant from selfing a heterozygous plant). Each of the pools of M₂ seed (i.e., all the seed from one pot or flat) are harvested together. These different pools are then screened for the phenotype of interest.

Ozone-Sensitivity

The anthropogenic air pollutant ozone (O₃) is a well documented cause of oxidative stress in plants. Ozone enters the plant through open stomata, and then presumably degrades into·O2-, H2O2, and OH▪ in the aqueous apoplastic environment (HEATH, 1994).

For ozone-sensitivity, M₂ seeds are planted out at a density of ˜250/6″ pot and then when the plants are 2 weeks old, they are treated with 250 parts per billion ozone for 8 hours. This treatment does not injure wild type Arabidopsis. After 24 hours, ozone-sensitive mutants are identified as those plants that have dead or damaged leaves. As ozone generates oxygen free radicals within the plant, it is not surprising that ozone-sensitive mutants (e.g., vtc1-1) are deficient in the antioxidant, ascorbic acid.

The mutants vtc2-3, vtc3-1 and vtc4-1 all appear to be somewhat O₃-sensitive, as O₃ exposure of each of these mutants leads to partial collapse of at least one leaf. Given that these AsA-deficient mutants have widely different O₃-sensitive phenotypes, there appears to be some factor(s) distinguishing one from another.

The wt Col-0 ecotype of Arabidopsis is quite tolerant to O₃, probably because these plants mount an effective antioxidant response. In contrast, the AsA-deficient mutant vtc1-1 is extremely O₃ sensitive, with visible injury including lesion formation, enhanced chlorosis, and/or tissue collapse. Severely injured leaves do not recover after the exposure, however, immature leaves emerging during the treatment are not visually injured, presumably due to a lack of fully functioning mature stomata.

To test the hypothesis that AsA is important for protection against O₃ injury, we examined the sensitivity of the vtc mutants and found a surprisingly wide range of response to this source of oxidative stress. Two-week-old vtc and wt plants were exposed to 400 ppb O₃ for 8 hours. Directly before this treatment, one set of plants was moved to a control chamber with very similar environmental conditions, but where O₃ was depleted by activated charcoal filtration. Photographs were taken of representative treated and control plants 16 hours after the end of the O₃ exposure, and tissue from the control plants was assayed for total AsA (FIG. 12). The different AsA-deficient mutant lines have varied O₃-sensitivities, sometimes even within an allelic series. The vtc1-1 mutant is very sensitive to O₃ damage. Consistent with our observation that vtc1-2 has the same mis-sense mutation as vtc1-1, this mutant exhibits a similar sensitivity. This injury is seen as total collapse and death of both cotyledons and fully expanded leaves. The different vtc2 mutants have highly varied O₃-sensitive phenotypes. vtc2-1 is as sensitive to O₃ as the vtc1 mutants, and vtc2-3 appears to be somewhat sensitive, as O₃ exposure of this mutant leads to partial collapse of at least one fully expanded leaf per plant. However, vtc2-2 is not visibly injured by this high dose of O₃, despite that fact that it contains a steady state level of AsA very similar to vtc2-1 (FIG. 12). As with vtc2-3, the mutants vtc3-1 and vtc4-1 are only slightly more O₃-sensitive than the wt.

Determining Loss of Conversion from Mannose to AsA in Identified Mutants

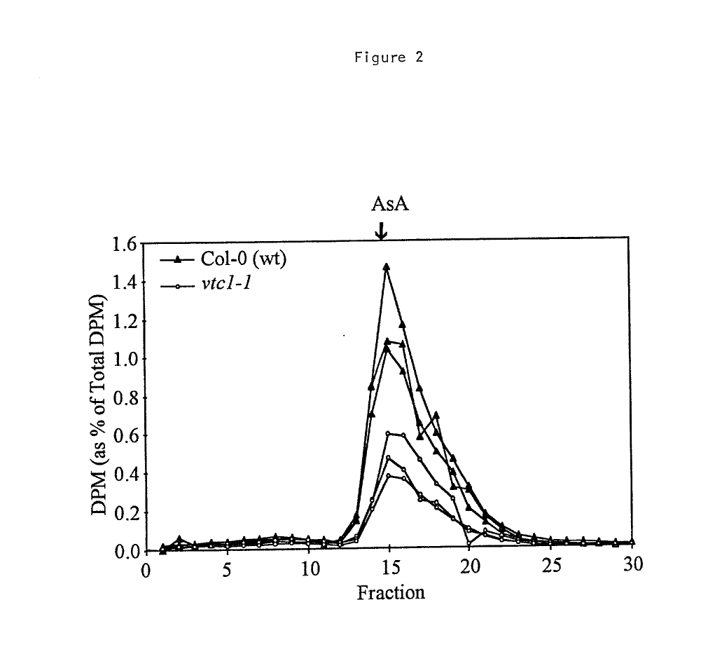

It is well established that D-glucose is a precursor to AsA, and previous results have shown that vtc1-1 is defective in the conversion of D-glucose to AsA. As D-mannose is a biosynthetic intermediate in the newly proposed pathway (FIG. 1), feeding studies were conducted to investigate whether vtc1-1 has a decreased ability to convert D-[U-¹⁴C] mannose to ¹⁴C-AsA. The labeling of vtc1-1 and wild type Col-0 leaves with D-[U-¹⁴C] mannose via the transpirational stream, fractionation of the labeled extracts, and further purification of L-[⁴C-AsA] by HPLC were done according to the methods of Wheeler et al., in Nature 393, 365-369 (1998), and Conklin et al, in Plant Physiol. 115, 1277-1285 (1997), the complete disclosures of which are hereby incorporated herein by reference. Briefly, excised leaves were fed with D-[U-¹⁴C] mannose through the transpirational stream for 1.5 hours and then transferred to water for 4 hours. AsA was fractionated from extracts of these labeled leaves and the amount of 1⁴C-AsA was then determined and expressed as a percent of 1⁴C in the total soluble fraction (FIG. 2). A greater percentage of 1⁴C was present as L-[1⁴C] AsA in wild type than vtc1-1 in every sample. Approximately 6.6% of the total ¹⁴C was present as L-[¹⁴C] AsA in the wild type samples compared to ˜2.6% in the vtc1-1 samples. Therefore, the AsA-deficient mutant vtc1-1 is defective in the conversion of D-mannose to AsA. These data strongly support the proposal that D-mannose is a substrate for AsA biosynthesis and that vtc1-1 is defective in one of the activities responsible for conversion of mannose to AsA.

Mapping the VTC Loci and Sequencing the VTC1 Gene

Each of the VTC loci were mapped onto the Arabidopsis genome by scoring genetic markers throughout the genome on vtc/vtc individuals (scored as NBT-) from a polymorphic F2 mapping population generated by a cross between the VTC/VTC (Ler ecotype) and vtc/vtc (Col-0 background). Both microsatellite and cleaved amplified polymorphic sequences were used as markers. VTC2, VTC3, and VTC4 were mapped using 50 vtc2-1/vtc2-1, 54 vtc3-1/vtc3-1, and 31 vtc4-1 /vtc4-1 F2 individuals. Genetic map locations were calculated using the Kosambi mapping function, which is well known in the art.

In order to determine the gene mutated in these AsA deficient plants, the VTC1 locus was mapped onto the Arabidopsis genome with 414 vtc1-1/vtc1-1 individuals developed from an F₂ mapping population derived from a cross with the Ler ecotype. Molecular markers used in this mapping included the cleaved amplified polymorphic sequence (CAPs) markers m429 and 178 and the microsatellite marker nga168.

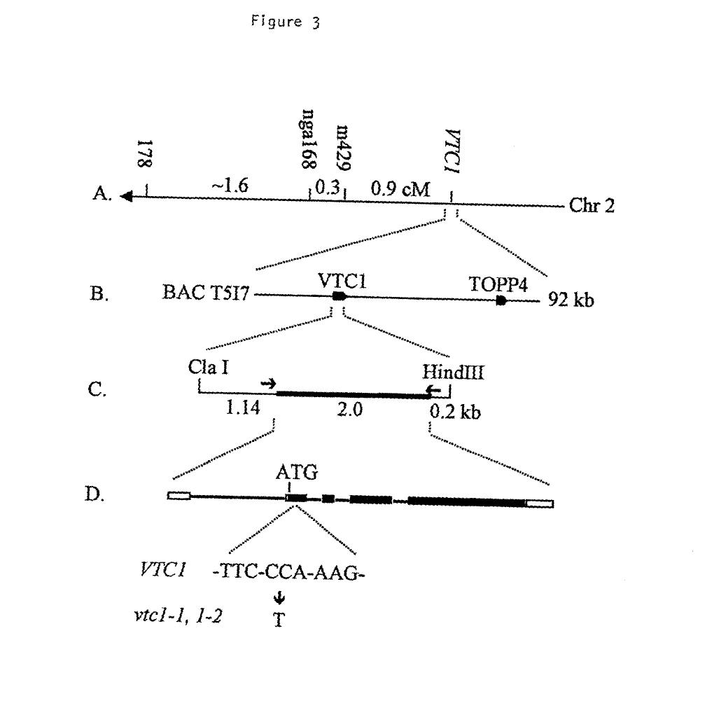

Using a mapping population of >400 F3 families derived from a cross between vtc1-1 and the wild type Ler ecotype, VTC1 was fine-mapped to a position on chromosome 2 to one side of two molecular markers; 0.9 cM from marker m429 and 1.2 cM from marker ngal 68 (as shown in FIG. 3A). Using microsatellite marker 178, which is >1 cM centromeric proximal to nga168, it was determined that VTC1 is centromere distal to nga168 and m429. All seven vtc1/vtc1 mapping lines that were recombinant between nga168 and VTC1 were also recombinant for marker 178 (including two between m429 and ngal 68), indicating that the relative order of these loci is as shown. This map is inconsistent with public domain recombinant inbred results, presumably because of the limited resolution of the recombinant inbred map: m429 is reported as being centromere proximal to nga168. See <http://nasc.nott.ac.uk/new_ri_map.html>. Our mapping data place VTC1 within a 2 Mb region on Chr 2 that spans m429 to just beyond marker m336, which is currently being sequenced by the Institute for Genomic Research (TIGR). The sequence of a 92 kb BAC (T5I7) within that contig (FIG. 3B) was annotated by TIGR, and the open reading frame T517.7 was identified as a putative mannose-l-phosphate guanyltransferase (www.tigr.org/docs/tigr-scripts/bac_{—}scripts/bac_display.spl?bac_name=T5I7). An alias for this enzyme is GDP-mannose pyrophosphorylase, which catalyzes step 4 in the proposed AsA biosynthetic pathway shown in FIG. 1. In this reaction, mannose-1-P is converted to GDP-mannose, with the consumption of GTP and the release of inorganic pyrophosphate (PPi).

Partial sequence for a GDP-mannose pyrophosphorylase cDNA, also annotated as encoding a putative mannose-1-phosphate guanyltransferase had been previously reported. The cDNA encoding the Arabidopsis GDP-mannose pyrophosphorylase (EST ID #9908, Genbank #T46645, www.ncbi.nlm.nih.gov/irx/cgi-bin/birx_doc?dbest_cu+6850) was obtained from the Arabidopsis Biological Resource DNA Stock Center (aims.cps.msu.edu/aims; Columbus, OH). This cDNA was fully sequenced on both strands. The sequence of a full-length cDNA encoding this protein defined all intron/exon borders, and this gene contains 5 exons with exon 1 and a small section of exon 2 being a 5′ untranslated region. The ˜40 kDa protein inferred from this open reading frame has 59% amino acid identity with the mannose-l-phosphate guanyltransferase from S. cerevisiae. The biochemical, molecular, and genetic evidence described herein supports the hypothesis that the VTC1 vitamin C biosynthetic locus encodes a GDP-mannose pyrophosphorylase.

To test the hypothesis that vtc1-1 and vtc1-2 harbor mutations in the GDP-mannose pyrophosphorylase gene, the potential for mutations in the pyrophosphorylase genomic sequence derived from each of these mutant alleles was examined. The sequences of both vtc1-1 and vtc1-2 contain the identical single cytosine to thymine point mutation at position +64 relative to the first base of the presumed initiator methionine (FIG. 3D). This predicted mis-sense mutation would convert a highly conserved proline to a serine at amino acid 22 in the GDP-mannose pyrophosphorylase amino acid sequence.

The point mutation in the vtc1 mutants does not alter the GDP-mannose pyrophosphorylase mRNA level. RNA filter hybridization analysis revealed no significant difference in the steady state level of the GMP-encoding MRNA in vtc1-1, vtc1-2 and wild type. These results are consistent with the hypothesis that the proline to serine change at amino acid position 22 affects the enzyme activity or stability, rather than transcription or MRNA stability.

The mutant alleles vtc1-1 and vtc1-2 were sequenced from PCR-amplification products of genomic DNAs. For each mutant allele, an ˜1.4 kb Bgl II fragment containing the majority of the coding region was sequenced using the primers, 5′ TGGTAAATACGCACTCAAT 3′ (SEQ ID NO: 1, named 5′-GMP) and 5′ AAAACAGCAAACGACCCTAACAA 3′ (SEQ ID NO: 2, named 3′-GMP). To confirm the public domain sequence of BAC T517 that included the base mutated in the vtc1 alleles, both strands of a portion of a Col-0 wild type VTC1 Cla I genomic clone (described below) were sequenced. The sequence of VTC1, vtc1-1, and vtc1-2 that included exon 1 and intron 1 was obtained directly from genomic DNA amplified with 5′-GMP and 5′ CATTCTTGTTGGAGGCTTCGG 3′ (SEQ ID NO: 3). The sequence downstream of the Bgl I fragment for vtc1-1 and vtc1-2 was obtained from genomic DNA amplified with the 5′ GAATAAGCATCAATCAAAACGC 3′ (SEQ ID NO: 4) and 5′ GCTAAGACCGACTTCAATCG 3′ (SEQ ID NO: 5). More than one independent PCR product was sequenced to confirm the veracity of the data.

Genetic Linkage and Segregation Analysis

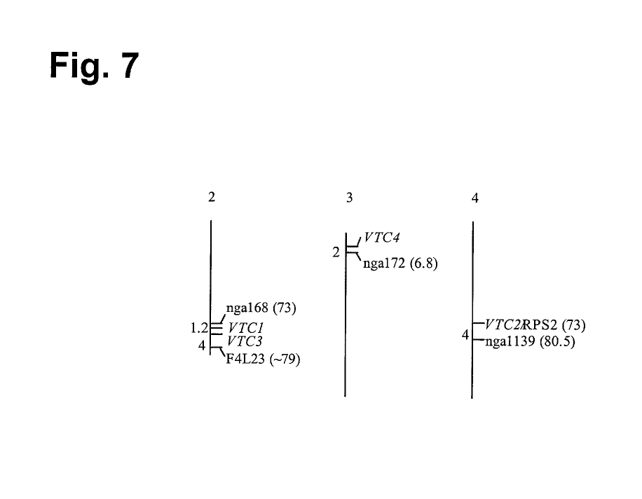

FIG. 7 shows map positions of the relative genetic map positions of the VTC1, VTC2, VTC3, and VTC4 loci on the Arabidopsis genome. Referring to FIG. 7, the numbers beside loci designations refer to approximate position in centiMorgans on the latest published RI map, while those at the top of the vertical lines indicate chromosome numbers. The VTC loci were mapped by scoring microsatellite or CAPs markers on vtc/vtc individuals from F2 polymorphic mapping populations segregating for vtc1-1, vtc2-1, vtc3-1, or vtc4-1. Using a mapping population segregating for either vtc2-1 or vtc2-2, VTC2 was fine-mapped to a position on Chr 4. Data from a mapping population generated for the vtc2-3 show that this mutant map to the same region as vtc2-1 and similarly vtc1-2 has been shown to map to the same position as vtc1-1. VTC3 was fine-mapped to Chr 2 and is closely linked to VTC1, ˜4 cM centromere proximal to a new microsatellite marker developed during this mapping that is located on BAC F4L23. With a small population, VTC4 was mapped to Chr 3. Genetic positions of the markers are from the latest recombinant inbred map and are shown in cM as are the distances between the VTC loci and a linked marker.

In addition to using NBT as a screening tool, the NBT assay was also used for segregation, linkage (Table 2), and mapping analyses of the vtc mutants. The AsA-deficiency in the mutants vtc1-2, vtc2-1, vtc2-2, vtc2-3, vtc3-1, and vtc4-1 are conferred by single monogenic recessive traits. F2 linkage analyses between the five newly isolated mutants and vtc1-1 and vtc2-1 clearly show that the vtc mutants represent four different loci: VTC1-VTC4.

To test whether the vtc mutation segregated as a single monogenic trait, F1 seed was obtained by pollination of VTC/VTC stigmas with vtc/vtc pollen or vice versa. Fl progeny were allowed to self-pollinate to obtain segregating F2 populations. Two-week-old plants from these populations were then scored using the NBT-based assay. To test for allelism, an F2 segregating population was obtained from a cross between two independently isolated ascorbic acid-deficient lines. Two-week-old F2 plants were then scored for AsA using the NBT-based assay. Two independently isolated vtc mutants were judged as non-allelic if F2 progeny with wt levels of AsA were obtained.

In addition to using the NBT-based assay to identify new mutants, it was also used for analyses of genetic segregation and allelism. In both cases, individual progeny from two independent crosses per mutant line were scored for the presence (NBT+) or absence (NBT-) of wt levels of AsA. Our data indicate that the AsA-deficiency in the mutants vtc1-2, vtc2-1, vtc2-2, vtc3-1 and vtc4-1 are conferred by single monogenic recessive traits. F2 progeny from crosses between three of the vtc mutant lines (vtc1-2, vtc2-2, vtc3-1) and wt Col-0 segregate in a statistically significant 3:1 ratio of NBT+: NBT− plants (p >0.2). In contrast, the F2 progeny from the cross between Col-O wt and vtc2-1 yielded an unexpectedly high number of NBT+individuals (p=0.003) while the F2 progeny of the cross between Col-0 and vtc4-1 included a somewhat high number of NBT− individuals (p <0.05; Table 1). These data are unlikely to result from a gene dosage effect, as both VTC2/vtc2-1 and VTC4/vtc4-1 heterozygotes contain wt levels of AsA. However, crossing both these mutant alleles to a different wt ecotype (Ler) yielded F2 progeny in the expected 3:1 ratio of NBT+:NBT-, suggesting that the AsA-deficiencies in these mutants are indeed conferred by single monogenic recessive traits.

The phenotypes of F2 progeny from crosses between the mutant vtc2-3 and Col-0 were somewhat skewed towards the presence of NBT− individuals. To test the hypothesis that this is a gene dosage effect, AsA levels were quantitatively measured in two sets of pooled F1 progeny from the cross (vtc2-3 x Col-0). As seen in FIG. 11, these F1 heterozygotes contain levels of AsA intermediate between the two parents, suggestive of a gene dosage effect. Given the fact that the NBT-based assay is only semi-quantitative, some of the VTC2/vtc2-3 F2 progeny were probably scored as NBT− resulting in the observed skewed ratio of NBT+/NBT− individuals.

We tested for allelism between the AsA-deficient Arabidopsis mutants and these results indicate that the vtc mutants represent four different loci: VTC1, VTC2, VTC3, and VTC4. Both wt (NBT+) and mutant (NBT−) individuals were found in the segregating F2 progeny from crosses between non-allelic mutants, such as vtc1-1 and vtc2-2. In contrast, the F2 segregating progeny from a cross between mutants harboring mutations at the same locus scored as mutant. A compilation of the segregation data shows that there are two vtc1 mutants and three vtc2 mutants, as well as single vtc3 and vtc4 alleles.

The F2 segregation data were extended by genetically mapping VTC2 through VTC4 (FIG. 7). The loci were mapped using polymorphic F2 mapping populations generated from crosses between these mutants and the wt line Ler, which contains well characterized microsatellite polymorphisms and cleaved polymorphic sequences (CAPS) compared with Col-0. The vtc2-1 mutation was found to map to a position on chromosome 4 approximately 3 cM centromere distal to CAPS marker WU95, which resides at position 71.70 on the latest Arabidopsis recombinant inbred (RI) genetic map (http://genome-www.stanford.edu/Arabidopsis/ww/Aug98RImaps/index.html) and approximately 5 cM centromere proximal to CAPS marker PRHA (position 76.17). vtc2-2 and vtc2-3 map to the same region as vtc2-1. VTC3 was mapped to a position on chromosome 2 close to VTC1, approximately 4 cM centromere distal from microsatellite marker nga168 (position 73.01). The VTC4 locus was mapped to the top of chromosome 4 approximately 2 cM centromere distal from microsatellite marker nga172 (position 6.83). VTC1 was previously mapped on chromosome 2 to a position 0.9 cM centromere distal from cleaved amplified polymorphic sequence (CAPS) marker m429.

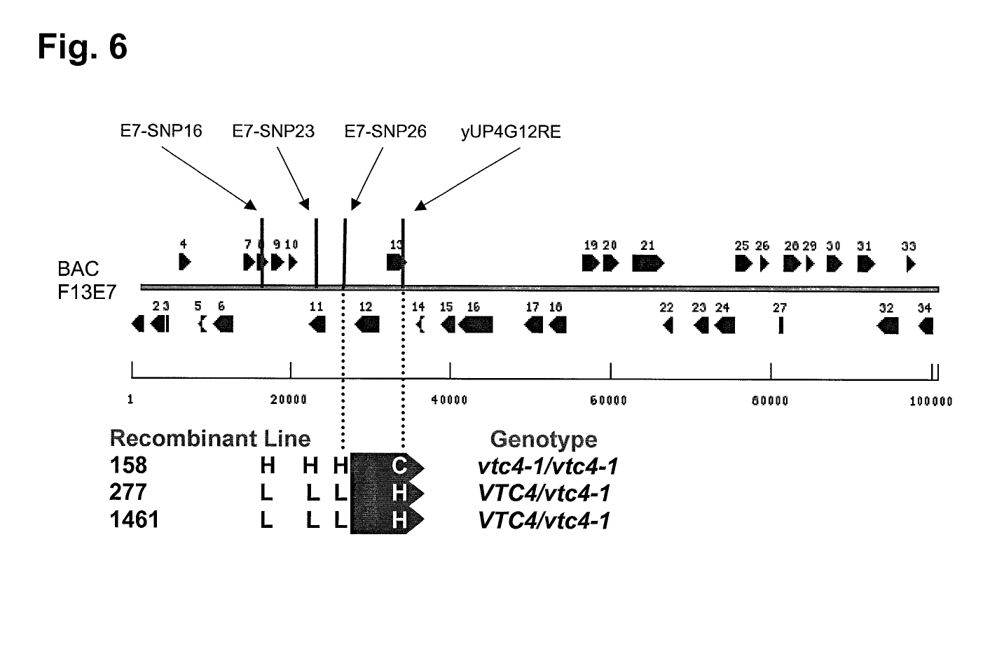

Referring to FIG. 6, F13E7.12 is annotated by the AGI as an “unknown protein” while F13E7.13 is annotated as a “putative replication factor A”

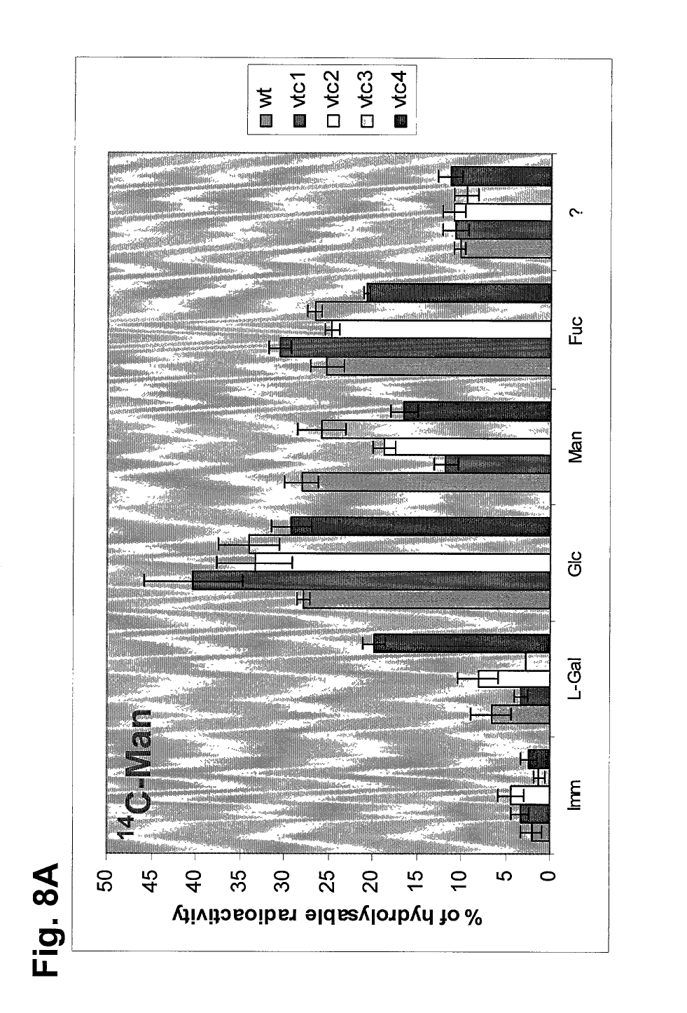

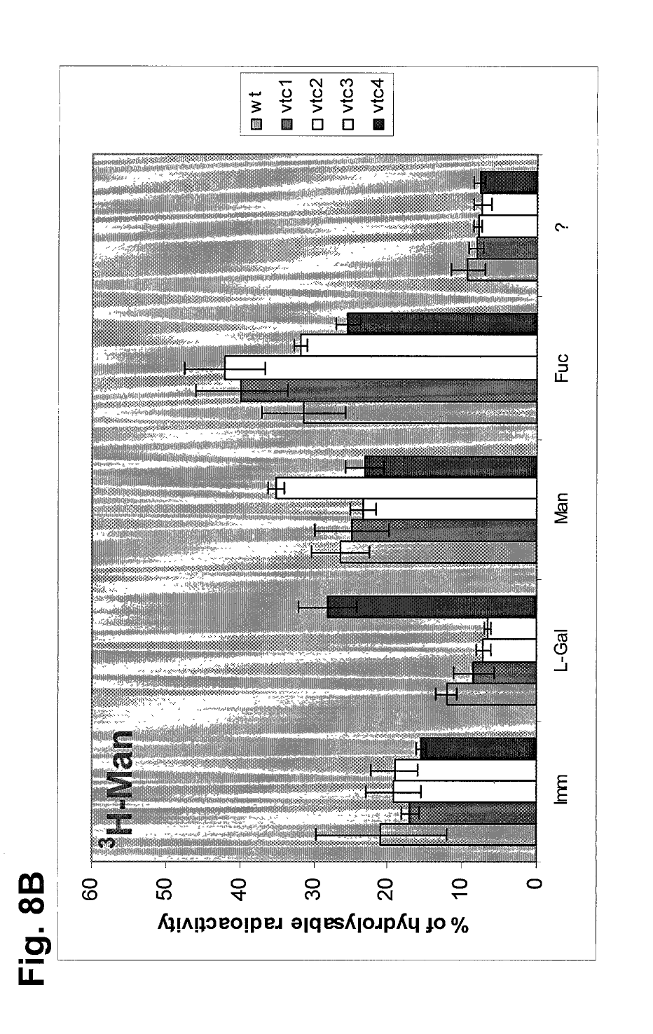

Referring to FIG. 8A and 8B, distribution of radioactivity in the TFA hydrolysable insoluble) fraction of Arabidopsis cell walls is shown. Detached leaves of wild type and mutant plants were supplied D-[U-14C]mannose or D-[2-3H]-mannose via the petiole for a total incubation time of 10 or 7 hours. The insoluble fraction was hydrolyzed with trifluoroacetic acid (TFA), the hydrolysate separated by TLC and the label determined in each sugar. Standard errors are shown (n-3). Abbrev. Imm, unhydrolysed polysaccharide and charged monomers; L-gal, L-galactose; Glc, glucose; Man, mannose; Fuc, fucose; ?, sum of all unidentified labeled compounds. In the 14C-mannose labeling, label can be converted to glucose-6-P (through PMI) which would then be metabolized to wall residues via UDP-glucose inter-conversions. In the 3H-mannose labeling, label in cell wall intermediates can only arise via GDP-mannose, as conversion to glucose-6-P via PMI would result in loss of label. As GDP-L-galactose is the form of L-galactose that is shunted to the cell wall, the accumulation of label in L-galactose in cell wall intermediates in vtc4-1 suggests that this mutant harbors a block in the pathway just downstream of GDP-L-galactose synthesis.

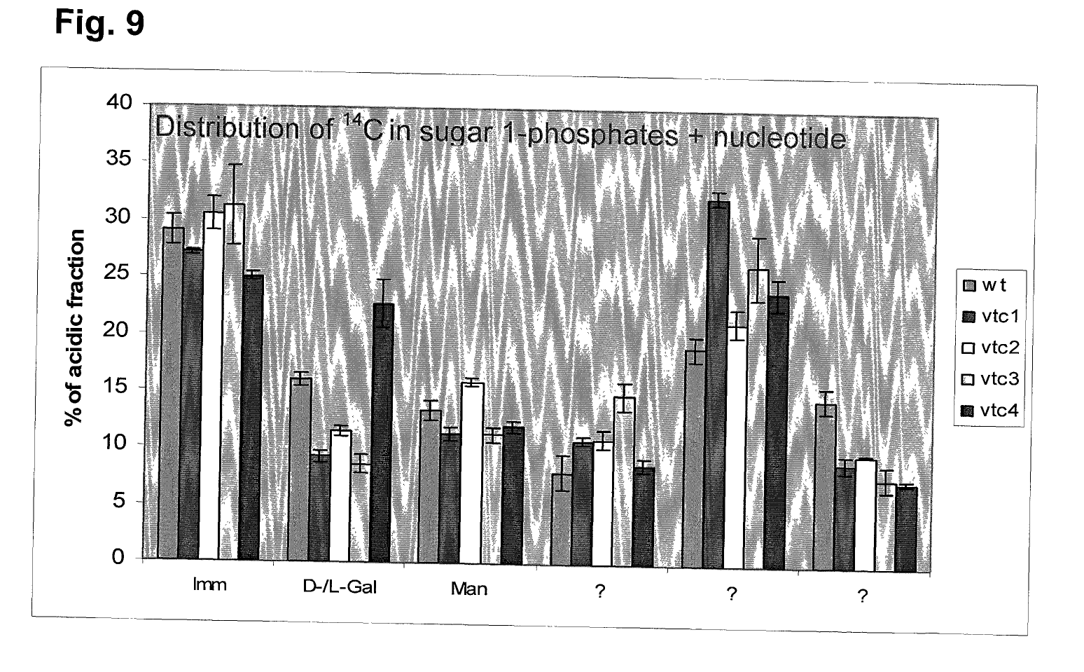

Referring to FIG. 9, the soluble acidic fraction from Arabidopsis leaves fed D-[U-14C]-mannose for 10 h was hydrolysed with TFA and the 14C content of sugars was determined. Values are means?? se (n=3). The soluble components derived from 14C-mannose fed leaves were fractionated by ion exchange, and the acidic (anionic) fraction was hydrolyzed and separated by TLC as above. The hydrolysis releases sugars that were present as NDP-sugars or 1-phosphates (6-phosphates are not hydrolyzed under these conditions and are immobile on the TLC plate). The results again show accumulation of label in galactose in vtc4-1, implying a relative accumulation NDP-Gal and/or Gal-1-P in the leaves. This is consistent with the wall data from slide 1; the interpretation being that vtc4 is blocked after GDP-L-Gal formation as increased availability of GDP-L-Gal would cause increased L-Gal incorporation into polysaccharide. Enzymatic analysis of vtc4-1 extracts reveal that L-Galactose-1-P phosphatase activity is normal, as are the activities of Phosphomannomutase and (most likely) Phosphomannose isomerase.

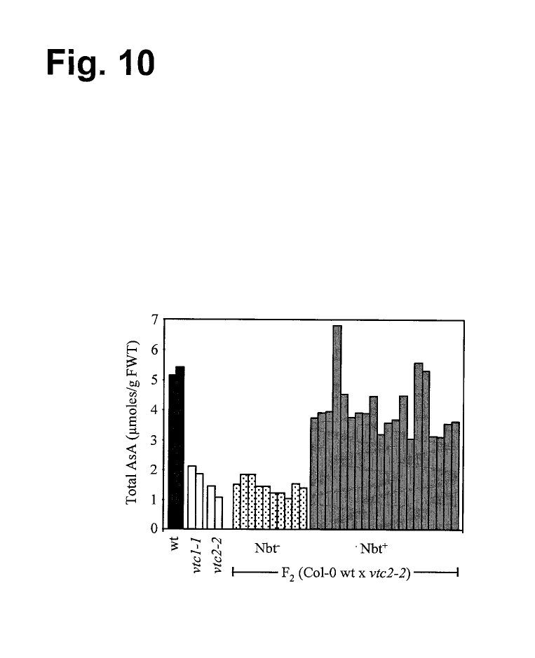

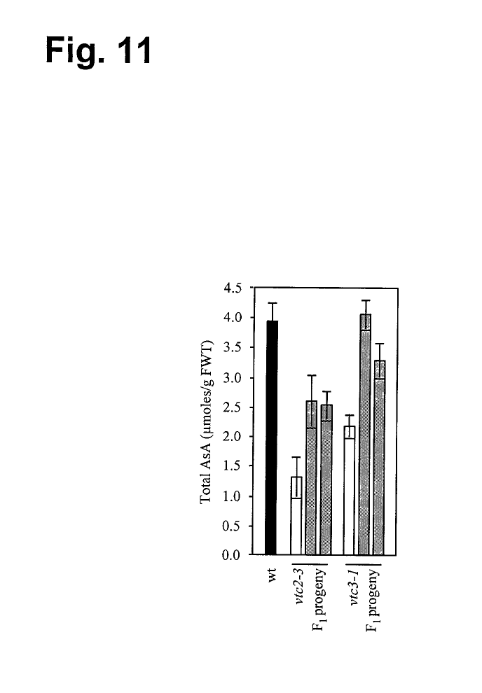

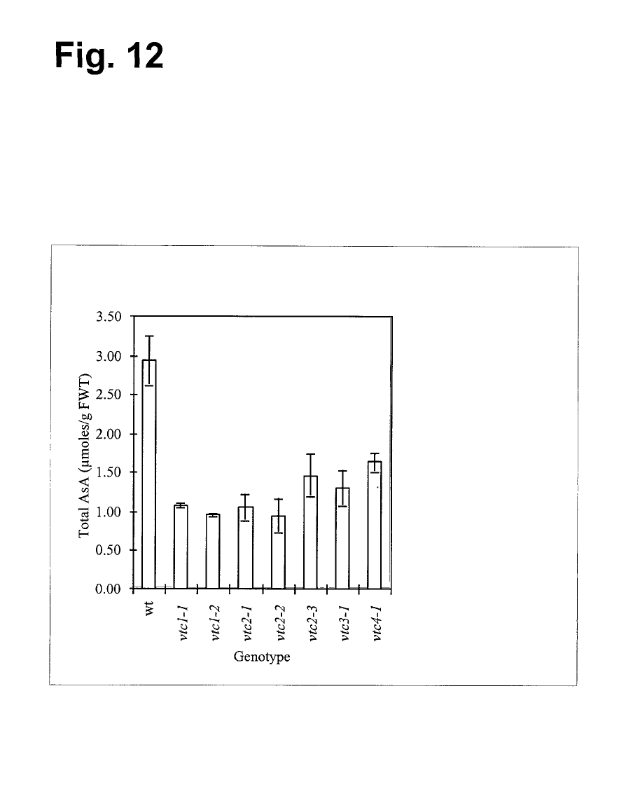

Referring to FIG. 10, the purple formazan precipitate in NBT-treated squashed leaves predicts AsA concentrations. One of the first two true leaves from two-week-old plants in an F2 population segregating for a vtc allele (Col-0 wt×vtc2-2) was scored for AsA using the NBT-based assay. The plants were then grown for an additional two days and entire rosettes from ten NBT− (little or no visible formazan) and 20 NBT+ (readily visible formazan) individuals and control plants (wt, vtc1-1, vtc2-2) were then harvested, extracted, and subjected to a quantitative total AsA assay using EC-HPLC.

Referring to FIG. 11, the VTC2 gene exhibits a gene dosage effect. Using a spectrophotometric assay, total AsA levels were measured in two-week-old wt, vtc2-3/vtc2-3, and VTC2/vtc2-3. Five individual rosettes were pooled for each extract and three independent extracts were assayed per sample with the mean and standard deviation shown. Assays were performed on Fl progeny from two independent crosses between Col-0 (wt) and the vtc2-3.

Referring to FIG. 12, quantitative analysis of total AsA levels in two-week-old vtc mutant and wt leaves. vtc and wt lines were spectrophotometrically assayed for total AsA. Whole rosette leaf tissue (50 mg) from pooled two-week-old plants was used for each extract. Three independent extracts were assayed per genotype with the mean and standard deviation shown. Each of the vtc lines used in this analysis was back-crossed (BC) to wt at least once to remove unlinked mutations (vtc1-1 was BC4; vtc1-2 and vtc4-1 were BC2; vtc2-1, vtc2-2, vtc2-3, vtc3-1 were BC1).

Referring to FIG. 13, the results of an analysis of AsA in vegetative and reproductive tissues of six-week-old vtc mutants and wt is shown. Samples of tissue (˜100 mg) from fully expanded mature leaves, developing siliques longer than ˜1 cm, and the influoresence (including opened and closed buds) were pooled from several individual six-week-old plants of each genotype. Extracts were prepared from these samples and assayed for total AsA as in FIG. 12. AsA levels in the different tissue types are indicated by the shaded boxes defined in the figure. The entire analysis was repeated with similar results (data not shown).

The profile of glycosylated protein from vtc3-1 and vtc4-1 is identical to wt. This suggests that vtc3-1 and vtc4-1 may not be deficient in GDP-mannose. Excised vtc3-1 and vtc4-1 leaves can convert both exogenous L-galactose and L-galactono-1,4-lactone to AsA at rates similar to wt, suggesting that the mutants are probably not defective in Steps 6 or 7 (FIG. 1). If the proposed pathway is correct and is the only functional plant AsA biosynthetic pathway, these experiments suggest that both mutants may be defective in a step(s) in the conversion of GDP-mannose to L-galactose.

As mentioned above, the two candidate VTC4 genes are annotated by AGI as (1) unknown protein (F13E7.12) and (2) putative replication factor A (F13E7.13). We propose that VTC4=F13E7.12. The radiolabeling experiments suggest that the vtc4-1 mutant is defective in the breakdown of GDP-L-galactose. It is difficult to resolve this function with a defect in DNA replication. The gene(s) encoding the biosynthetic enzyme(s) involved in the conversion of GDP-L-galactose to L-galactose have not been identified in other organisms, therefore such gene(s) would presumably be annotated as encoding “unknown proteins” in the database.

The F13E7.12 protein has similarity to two other predicted Arabidopsis proteins, F5E19.70 (71.9% identity), and F13011.30 (45.8% identity), as well as a predicted maize protein (EST AI621709: 38.3% identity). The predicted Arabidopsis proteins are all annotated by the AGI as “unknown proteins”. No other proteins in the NCBI database were found to have significant similarity to F13E7.12.

The ascorbic acid deficient mutant vtc4-1 appears to be defective in conversion of GDP-L-galactose to galactose-1-P. Radiolabeling experiments indicate that this mutant accumulates GDP-L-galactose in both soluble and cell wall polysaccharides. This mutant has normal conversion of galactose-1-P to L-galactose, suggestive of a block in the conversion of GDP-L-galactose to galactose-1-P.

Determining GDP-Mannose Pyrophosphorylase Activity in the Mutant Plants

If the GDP-mannose pyrophosphorylase is mutated in this recombinant plant, then its activity should be impaired. To test this possibility, GDP-mannose pyrophosphorylase activity was assayed in the reverse direction in crude extracts that were prepared by extraction of 0.3 g of leaf tissue in 1 ml of 100 mM Tris pH 7.6, 1% PVP, 5 mM DTT, 1 mM EDTA followed by centrifugation to remove insoluble material. The reactions were performed by adding 30 ml of crude extract to 104 ml of 15.4 mM MgCl₂, 15.4 mM NaPPi, 13.5 mM Tris/HCI, pH 8.0, 1.1 mM EDTA and 0.1 μCi GDP-[¹⁴C]-mannose (Amersham, UK), and were terminated by boiling. The reactions were clarified by centrifugation and then lyophilized. For separation of the nucleotide sugars from the sugar phosphates by thin layer chromatography, the samples were resuspended in dH₂0 and a fifth of each sample was spotted onto cellulose plates (150 μm, K2 cellulose, Whatman, Clifton, N.J.). The separation solvent was ethanol/1 M ammonium acetate, pH 5.0 (60:40 by volume).

To detect radioactivity, the thin layer chromatography plates were scanned with a Berthold Linear Analyzer (Berthold LB2832, Hemstead, U.K.). The identification of nucleotide sugars and sugar phosphates were determined first by comparison to a co-migrating GDP-[¹⁴C]-mannose standard and second by staining plates with an ammonium molybdate stain by the said technique being known in the art, and incorporated by reference (Dawson, R. M. C. et al. (1986) in Data for Biochemical Research, Third Edition, Oxford Univ. Press, London, pp. 485-486). The nucleotide sugars and sugar phosphates were scrapped off the cellulose plates and eluted from the cellulose in dH₂0. The free sugars were released by hydrolysis and analyzed as described in Wheeler, G. L. et al. (1998) Nature 393, 365-369. Protein concentrations were determined by the Bradford assay with γ-globulin as a control.

The Arabidopsis leaf extracts contained a potentially interfering phosphodiesterase activity that produced mannose-1-P and GMP from GDP-mannose. However this phosphodiesterase activity was completely inhibited by the high PPᵢ concentration used in the pyrophosphorylase assay. This inhibition of phosphodiesterase by PPᵢ was confirmed by experiments with bovine intestinal mucosa phosphodiesterase 1 (Sigma, St. Louis, Mo.) under the same conditions as the pyrophosphorylase assay.

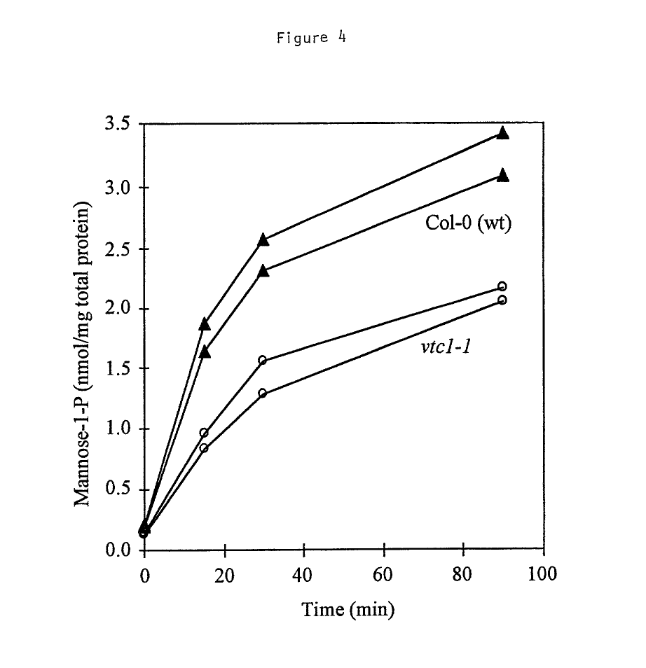

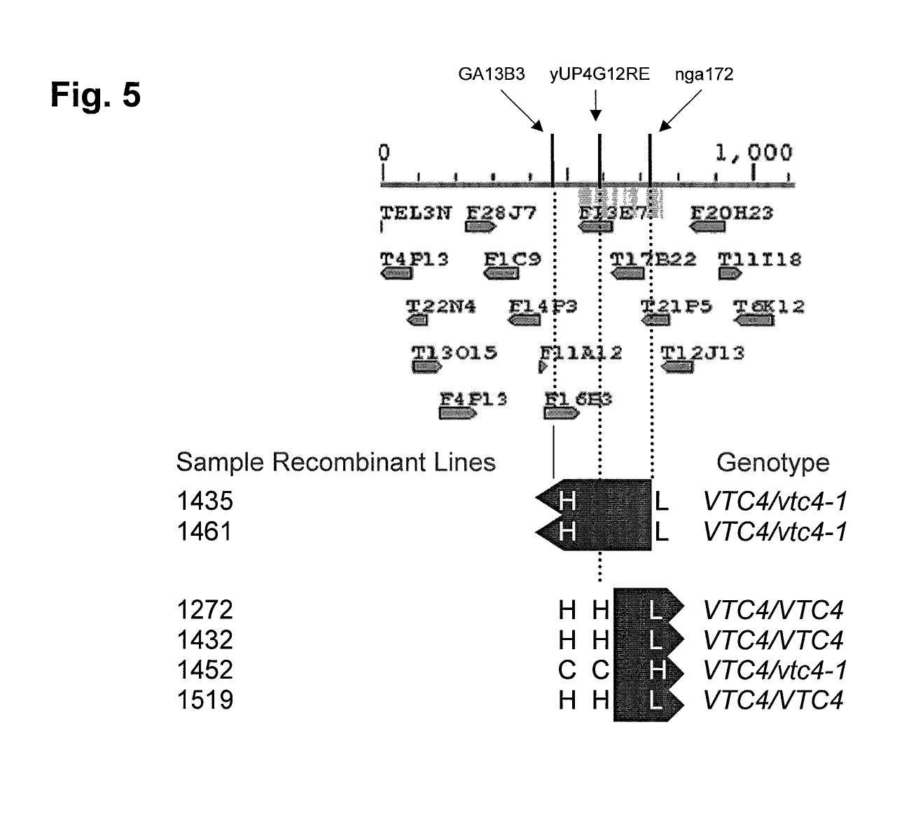

If VTC1 encodes GDP-mannose pyrophosphorylase, the AsA-deficient mutant vtc1-1 would be predicted to have reduced enzyme activity compared with wild type plants. As the activity of this enzyme is fully reversible in vitro, pyrophosphorylase activity can be assayed by monitoring the production of mannose-1-P from GDP-mannose and PPi by the said technique being known in the art, and incorporated by reference (Szumilo, T. et al. (1993) J. Biol. Chem. 268, 17943-17950). This assay was used to measure GDP-mannose pyrophosphorylase activity in extracts from both vtc1-1 and wild type. The time-dependent production of mannose-1-P from GDP-mannose and PPi is lower in extracts from vtc1-1 than wild type. After a 90 minute incubation, ˜35% less mannose-l-P is formed in vtc1-1 compared to wild type (FIG. 4).

Rescue of the Mutant Phenotype by Creating A Recombinant Plant

By introducing the wild type version of the GDP-mannose pyrophosphorylase gene, the mutant phenotype should be rescued. In effect, the recombinant plant created via transformation will be able to functionally express recombinant GDP-mannose pyrophosphorylase and restore function.

A 5.4 kb Cla I fragment containing the VTC1 locus was subcloned from BAC T517. A 3.4 kb fragment from this subclone was then ligated into the binary vector pGPTV-BAR/Hin dIII by the said technique being known in the art, and incorporated by reference (Becker, D. et al. (1992) Plant Mol. Biol. 20, 1195-1197). This construct (gVTC1-pGPTV) was transformed into Agrobacterium tumefaciens pMP90 strain GV3101 and introduced into vtc1-1 plants by vacuum infiltration.

The vacuum filtration method for transformation is discussed below. The seeds are planted on top of window screen covered soils. After the plants have bolted, clip off the primary bolt to encourage growth of secondary bolts. Perform infiltration around four days after clipping. Start a 20 ml overnight culture of Agrobacterium carrying the gVTC1-GPTV construct including the appropriate antibiotics (kan, rif, and gm) two days prior to transformation. The day before the transformation, use this overnight culture to inoculate a large (˜500 ml) culture. After 24 hours of growth, harvest cells by centrifugation and wash once with growth media without antibiotics. Resuspend bacteria at 0.8 OD units in infiltration media. One liter of infiltration media consists of 0.5×MS salts, 1×B5 vitamins, 5% sucrose, 0.044 uM benzylamino purine, 0.03% Silwet L-77, and 0.5 g MES (pH to 5.7 with KOH). Pour some of diluted bacteria into a Rubbermaid™ dish that fits inside the vacuum oven (be sure to turn oven temperature off prior to use). Invert pot with plants to be infiltrated into culture and place in vacuum oven. Infiltrate 5-10 min at 15 in ³Hg. The vacuum is not necessary as just dipping the plants into the culture for ˜5 min also gives similar transformation frequency. For the p VTC1-pGPTV infiltrations, both vacuum infiltration and dipping alone produced similar results. Release the vacuum and remove the pot. Cover with plastic wrap and return to the light room. Remove the cover the next day. A newer streamlined procedure being known in the art, and incorporated by reference (S. J. Clough and A. F. Bent, 1998. Plant J. 16:735-743) can alternatively be used for transformation.

Glufosinate-ammonium resistant T₁ transgenic individuals were selected by sowing seeds and spraying the soil surface with 500 ml per m² of 0.25 mg ml⁻¹ commercially formulated glufosinate-ammonium (Finale; AgrEvo, Montvale N.J.). Twelve days after sowing, resistant T₁ seedlings were transplanted to non-treated soil and allowed to self-pollinate.

T₂ progeny were scored for glufosinate-ammonium resistance by painting individual leaves with the herbicide (150 μg ml⁻¹ glufosinate-ammonium, 250 nl ml⁻¹ Silwet). These plants were also scored for wild type or mutant (deficient) levels of AsA by a nitroblue tetrazolium-based method in which single leaves are squashed onto chromatography paper and treated with 1 mg/ml of nitroblue tetrazolium. The AsA in wild type leaves is sufficient to reduce the nitroblue tetrazolium to the visible precipitate formazan, while no readily visible formazan is produced upon treatment of vtc1-1 leaves (Conklin et al., in preparation). AsA levels were then confirmed by a previously described spectrophotometric-based assay (Conklin, P. L. et al. (1996) Proc. Natl. Acad. Sci. USA 93, 9970-9974).

If the VTC1 locus encodes GDP-mannose pyrophosphorylase, a wild type copy of this locus introduced as a transgene will complement the vtc1-1 allele and restore normal levels of AsA. To test this hypothesis, a genomic clone including ˜1.1 kb upstream of the 5′ end of the GDP-mannose pyrophosphorylase cDNA and 0.2 kb downstream of the predicted stop codon (FIG. 3c) was subcloned from BAC T5I7 and transformed into vtc1-1 plants by the Agrobacterium tumefaciens vacuum infiltration method. T₁ transgenic plants were selected by glufosinate-ammonium resistance conferred by the BAR gene.

TABLE 1
Cosegregation of elevated AsA levels and the selectable marker in vtc1-1
lines transformed with genomic copy(s) of the VTC1 locus.

| Line | AsA + ¹(# Basta^{R2}/total) | AsA − (# Basta^{R}/total) |
|---|---|---|
| 1 | 79 (10/10) | 28 (1/11) |
| 2 | 70 (10/10) | 34 (0/12) |
| 3 | 75 (11/11) | 29 (0/10) |

Thirteen glufosinate-ammonium resistant T₁ transgenics that were confirmed to contain the BAR gene by PCR-mplification all contained wild type levels of AsA. These results were consistent with the hypothesis that the transgene complemented vtc1-1. The T₁ lines were allowed to self-pollinate and three selected T₂ lines from independent T₁ lines were tested for co-segregation of wild type levels of AsA (scored using a qualitative AsA assay) and glufosinate-ammonium resistance. Introduction of the VTC1 locus into the AsA-deficient vtc1-1 mutant confers increased levels of AsA that co-segregate with the selectable marker (Table 1). Finally, ten individuals that scored as wild type for AsA from each ₂ line were pooled, extracts were prepared, and total AsA was measured using a quantitative spectrophotometric assay. These pooled extracts contained between 2.4 and 3.8 μmoles AsA/g FWT of AsA which is similar to the 3.1 μmoles AsA/g FWT seen in wild type, and greater than the 0.9 μmoles AsA/g FWT in the mutant. Together, these results confirm that the VTC1 locus encodes a GDP-mannose pyrophosphorylase structural gene.

Applications of the Technology

GDP-mannose pyrophosphorylase is an enzyme in the recently proposed plant AsA biosynthetic pathway (FIG. 1). This invention provides conclusive evidence that GDP-mannose pyrophosphorylase is encoded by the VTC1 locus in Arabidopsis, and that the enzyme is a critical component of the AsA biosynthetic pathway. First, the AsA-deficient vtc1-1 mutant is defective in the conversion of mannose to AsA. Second, the activity of GDP-mannose pyrophosphorylase is lower in extracts from vtc1-1 than wild type. Third, the VTC1 locus genetically maps to a region of genomic DNA encoding a GDP-mannose pyrophosphorylase homologue and the vtc1-1 and vtc1-2 mutants each harbor the identical point mutation that alters a highly conserved proline residue in this gene. Finally, a transgene encoding the wild type pyrophosphorylase genetically complements the vtc1-1 mutation, increasing the AsA in the transgenic vtc1-1 lines to levels similar to wild type. These results demonstrate that the AsA biosynthetic pathway proposed based on in vitro biochemical data operates in vivo.

The AsA-deficient Arabidopsis mutants isolated are unique and ideal tools for the testing of this pathway. The VTC1 locus described by one of these AsA-deficient mutants has been cloned here. As the first genetically identified plant AsA biosynthetic gene, VTC1 has already proved the efficacy of this approach. Armed both with the knowledge of this proposed pathway and AsA-deficient mutant lines, other biosynthetic genes can be readily isolated and characterized.

There is existing evidence to suggest that increasing the AsA content of plants will be advantageous for protection against environmental sources of ROS. The AsA-deficient mutant vtc1 is highly sensitive to O₃, a potent generator of ROS in the plant. This sensitivity can be abolished by treatment of the mutant with exogenous AsA prior to the start of the fumigation (Conklin, P. L., et al. 1996. Proc Natl Acad Sci USA 93: 9970-9974). This pretreatment increases the concentration of AsA in vtc15-20X. Similarly, the AsA levels can also be raised in wild type Arabidopsis at least 5-6X. Increasing the AsA level in the plant also abolishes the O₃-sensitivity of soz (sensitive to ozone) mutants that synthesize wild type levels of AsA. Therefore, increased AsA levels have the capacity to cross-protect lines with sensitivities not correlated to an AsA-deficiency. In the literature there are several other examples of a correlation between artificially increased AsA and decreased sensitivity to O₃ including one published almost four decades ago. In this experiment, a sensitive tobacco variety (Bel-W3) that is not AsA-deficient was pretreated with AsA prior to O₃ fumigation. This pretreatment increased the AsA level in this sensitive variety and decreased its O₃ sensitivity (Menser, H. A., 1964. Plant Physiol 39: 564-567). In a more recent study, pretreatment of barley leaves with AsA protected both plasma membrane permeability and the light regulation of ribulose-1,5-bisphosphate carboxylase-oxygenase (rubisco) from O₃ damage (Machler, F., et al., 1995. J Plant Physiol 147: 469-473.). Studies with the air pollutant sulfur dioxide have also shown a positive relationship between application of exogenous AsA and increased resistance to this source of ROS (Pandya, N., and S. J. Bedi, 1990. Adv Plant Sci 3: 171-177). Since increased AsA clearly protects sensitive varieties from the ROS produced from air pollutants such as O₃ and sulfur dioxide, the present invention will be crucial in the development of tools for manipulating increased AsA levels.

The identification of genes involved in plant AsA biosynthesis provides us with tools to increase the endogenous AsA levels in transgenic plants. Over-expression of Arabidopsis GDP-mannose pyrophosphorylase in plants where this enzyme is a limiting factor results in increased synthesis of GDP-mannose, a key intermediate in AsA biosynthesis. VTC1/vtc1 heterozygotes exhibit a gene dosage effect, having intermediate levels of AsA. This shows that the GDP-mannose pyrophosphorylase activity is limiting for AsA biosynthesis. Over-expression of VTC1 in plants results in increased AsA levels. In addition to having increased nutritive value, such transgenic plants will have increased resistance to a number of environmental stresses.

The teachings of the present invention can be used as tools for use in improving the nutritional quality and environmental stress resistance of agronomically important plants as well as serving as plant-specific herbicide targets. Increased environmental stress tolerance alone could result in economic benefits from increased yield as many common adverse conditions including drought, chilling, high light, heavy metals, UV-B, and air pollutants produce damaging ROS. Basic plant metabolic pathways are normally highly conserved among different plant species. If AsA levels can be increased by over-expression of AsA biosynthetic genes in Arabidopsis, the technology is readily transferable to agronomically important crop plants by known methods in the art.

Accordingly, it is to be understood that the embodiments of the invention herein described are merely illustrative of the application of the principles of the invention. Reference herein to details of the illustrated embodiments are not intended to limit the scope of the claims, which themselves recite those features regarded as essential to the invention.

What is claimed is:

1. A genetically engineered plant, or portion thereof, comprising a recombinant nucleic acid sequence that encodes a protein involved in Vitamin C biosynthesis.

2. The genetically engineered plant of claim 1 wherein said plant, or portion thereof, is a dicot.

3. The genetically engineered plant of claim 1 wherein said genetically engineered plant is Arabidopsis thaliana.

4. The genetically engineered plant, or portion thereof, of claim 1 wherein said nucleic acid comprises a polynucleotide that encodes GDP-mannose pyrophosphorylase, or a polynucleotide that encodes a VTC4 gene product.

5. The genetically engineered plant of claim 1 wherein said genetically engineered plant, or portion thereof, is capable of over-expressing said recombinant nucleic acid.

6. The genetically engineered plant of claim 1 wherein said genetically engineered plant, or portion thereof, is capable of producing increased levels of Vitamin C.

7. The genetically engineered plant of claim 1 wherein said genetically engineered plant, or portion thereof, has increased resistance to environmental stress compared to a plant of the same species without said recombinant nucleic acid wherein said environmental stress is selected from the group consisting of:
a) drought;
b) cold;
c) UV radiation;
d) air pollution;
e) salts;
f) heavy metals; and
g) reactive oxygen species.

8. The genetically engineered plant of claim 1 wherein said genetically engineered plant, or portion thereof, is edible.

9. A genetically engineered plant, or portion thereof, comprising a recombinant nucleic acid that encodes GDP-mannose pyrophosphorylase, or a recombinant nucleic acid that encodes a VTC4 gene product.

10. The genetically engineered plant of claim 9 wherein said genetically engineered plant, or portion thereof, is a dicot.

11. The genetically engineered plant of claim 9 wherein said genetically engineered plant is Arabidopsis thaliana.

12. The genetically engineered plant of claim 9 wherein said genetically engineered plant, or portion thereof, is capable of over-expressing said recombinant nucleic acid.

13. The genetically engineered plant of claim 9 wherein said genetically engineered plant, or portion thereof, is capable of producing increased levels of Vitamin C.

14. The genetically engineered plant of claim 9 wherein said genetically engineered plant, or portion thereof, has increased resistance to environmental stress compared to a plant of the same species without said recombinant nucleic acid wherein said environmental stress is selected from the group consisting of:
a) drought;
b) cold;
c) UV radiation;
d) air pollution;
e) salts;
f) heavy metals; and
g) reactive oxygen species.

15. The genetically engineered plant of claim 9 wherein said genetically engineered plant, or portion thereof, is edible.

16. A method of increasing the endogenous level of Vitamin C produced in a plant, or portion thereof, comprising over-expression of an enzyme crucial to Vitamin C biosynthesis.

17. The method of claim 16 wherein said enzyme is GDP-mannose pyrophosphorylase, or a VTC4 gene product.

18. The method of claim 16 wherein said plant, or portion thereof, is a dicot.

19. The method of claim 16 wherein said plant is Arabidopsis thaliana.

20. The method of claim 16 wherein said plant, or portion thereof, comprises increased anti-oxidation capacity.

21. The method of claim 16 wherein said plant, or portion thereof, has increased resistance to environmental stress compared to a plant of the same species without said recombinant nucleic acid wherein said environmental stress is selected from the group consisting of:
a) drought;
b) cold;
c) UV radiation;
d) air pollution
e) salts;
f) heavy metals; and
g) reactive oxygen species.

22. The method of claim 16 wherein said method produces a plant, or portion thereof, which is edible.

23. A genetically engineered plant comprising a mutant gene that encodes a form of GDP-mannose pyrophosphorylase, or a mutant gene that encodes a VTC4 gene product.
